(19) United States
(12) Patent Application Publication
(10) Pub. No.: US 2010/0196292 A1
(43) Pub. Date: Aug. 5, 2010

(54) Hydrophylic silicone elastomers in cosmetics

(75) Inventors: John C. Carson, Union City, NJ (US); Nagi M. Awad, Franklin Lakes, NJ (US); Albert A. Zofchak, Holmdel, NJ (US); Michael Mosquera, Forked River, NJ (US)

Correspondence Address: Henry D Coleman, 714 Colorado Avenue, Bridgeport, CT, 06605-1601, US

(73) Assignee: ALZO INTERNATIONAL, INC., Sayreville, NJ (US)

(21) Appl. No.: 12/657,850

(22) Filed: Jan. 28, 2010

Related U.S. Application Data
(60) Provisional application No. 61/206,106, filed on Jan. 28, 2009.

(51) Int. Cl. A61K 8/89; A61K 8/92; A61K 47/34; A61Q 1/02; A61Q 5/02; A61Q 15/00; A61Q 5/12; A61Q 17/04; C08G 77/38; A23L 1/00

(52) U.S. Cl. 424/59; 424/65; 424/63; 424/70.12; 514/772.3; 525/453; 525/474; 525/479; 426/654; 512/4

(57) ABSTRACT

The invention is directed to the use of a number of hydrophilic silicone elastomers in various cosmetic and personal care products where they serve various functions such as emulsifiers, film forming agents, viscosity stabilizers, conditioning agents and protective barrier forming agents. Emulsions and personal care products based upon same are also described.

### RELATED APPLICATIONS

This application claims the benefit of priority of U.S. provisional application Ser. No. 61/206,106, filed January 2009, of identical title, which is incorporated by reference in its entirety herein.

### FIELD OF THE INVENTION

The invention is directed to the use of a number of hydrophilic silicone elastomers in various cosmetic and personal care products where they serve various functions such as emulsifiers, film forming agents, viscosity stabilizers, conditioning agents and protective barrier forming agents. Emulsion compositions according to the present invention are also disclosed.

### BACKGROUND

Elastomeric silicone polymers are well known materials in the cosmetic and personal care industries. These materials are composed of dimethylsiloxane polymers that are crosslinked into large molecular entities that are capable of forming films, increasing and stabilizing viscosity, altering product rheology, entrapping water soluble component powders (such as ascorbic acid powder) and, most significantly for cosmetic products, they greatly improve the smoothness and uniformity of product application and add a feeling of elegance. Crosslinked silicone elastomers of particular interest are those described in U.S. Pat. No. 6,936,686 which teaches the preparation and use of silicone elastomers that are crosslinked as a solution in either a low viscosity silicone oil, a hydrocarbon oil (such as isododecane), a cyclomethicone or mixtures thereof. Such silicone elastomers are typically compatible with many of the cosmetic esters, polydimethylsiloxanes and aliphatic compounds used in cosmetic and personal care products. However, they are not at all compatible with water or water soluble ingredients and, while this property contributes to their ability to be successfully emulsified, it does limit their utility in cosmetic products.

U.S. Pat. No. 6,936,686 also teaches the use of an alkylene ethoxylate as a co-reactant to increase the hydrophilicity of the resulting elastomer. Such hydrophilic elastomers find great utility as water-in-oil (w/o) emulsifiers, as protectants and carriers for other water soluble ingredients and as fully functional silicone elastomers having the expected properties described previously.

In addition to U.S. Pat. No. 6,936,686, other compositions which relate to the formation of hydrophilic silicone elastomers from the admixture of a traditional hydrophobic silicone elastomer with a hydrophilic polyurethane or the chemical reaction product of a silicone elastomer with an alkene containing hydrophilic polyurethane. These hydrophilic polyurethane silicone elastomers provide additional solubility opportunities (because the polyurethane solubility characteristics can be widely varied i.e. increased hydrocarbon solubility), controllable hydrophilicity and variable deposition capacity (because the polyurethane can contain an amine or quaternary ammonium salt).

The use of these hydrophilically modified silicone elastomers can produce cosmetic products such as water-in-oil-in-water (w-o-w) emulsions that can be simply prepared using conventional equipment. Hydrophilically modified silicone elastomers allows the use of water and water soluble ingredients in formulating cosmetic products that are typically anhydrous such as lipsticks, lip glosses and silicone elastomer based makeup products. Hydrophilically modified silicone elastomers can also be used in hair care products such as hair conditioners, hair setting products, shampoos and hair shine products. Thus, the product application possibilities are greatly expanded through the use of the hydrophilically modified silicone elastomers that are taught by U.S. Pat. No. 6,936,686 and herein.

### OBJECTS OF THE INVENTION

The present invention relates to the use of hydrophilically modified silicone elastomers that are taught by U.S. Pat. No. 6,936,686 and related compositions in cosmetic and personal care products.

It is an object of this invention to use the hydrophilically modified silicone elastomers that are taught by U.S. Pat. No. 6,936,686 and otherwise as described herein in personal care and cosmetic emulsions to provide emulsification, emulsion stabilization, film forming properties and the ability to form water-in-oil-in-water emulsions. Examples of these products include hand, body and face lotions and creams, sunscreen products, antiperspirants, depilatories, hair colors, developers and anti-acne preparations.

It is an object of this invention to use the hydrophilically modified silicone elastomers that are taught by U.S. Pat. No. 6,936,686 and herein in personal care and cosmetic products that contain pigments to act as a dispersing, film forming component and emulsifying agent. Examples of these products include liquid and cream make-up products, mascaras, eyeliners, blushes and rouges.

It is an object of this invention to use the hydrophilically modified silicone elastomers that are taught by U.S. Pat. No. 6,936,686 and herein in anhydrous personal care and cosmetic products to facilitate the addition of water to those products. These typically anhydrous products are represented by, but not limited to, the examples of lipstick, lip gloss, ointments, hair shine or glossing products, personal fragrances, make-up liquids, make-up powders, make-up pressed powders, make-up poured powders and cover sticks.

It is an object of this invention to use the hydrophilically modified silicone elastomers that are taught by U.S. Pat. No. 6,936,686 and herein in hair care products such as hair conditioners, hair setting products, hair protectants and hair shine products.

It is an object of this invention to use the hydrophilically modified silicone elastomers that are taught by U.S. Pat. No. 6,936,686 and herein in personal cleaning products such as hair shampoos, body shampoos, soaps, liquid soaps and facial washes.

It is an object of this invention to use the hydrophilically modified silicone elastomers that are taught by U.S. Pat. No. 6,936,686 and herein in personal care and medical products to provide a method for the containment and subsequent release or reaction of water soluble or water activated components. Examples of these concepts include the emulsification of a dihydroxyacetone solution in a gel matrix comprised of the hydrophilically modified silicone elastomers that are taught by U.S. Pat. No. 6,936,686 and herein and additional suitable anhydrous vehicles and the containment of anhydrous Zeolite in the described hydrophilically modified silicone elastomers that produces measurable heat upon the addition of water.

Any one or more of these and/or other objects of the invention may be readily gleaned from a review of the description of the invention which follows.

### BRIEF DESCRIPTION OF THE FIGURES

FIGS. 1-6 show representative polyurethanes which may be reacted with polyorganosiloxanes according to the present invention to provide hydrophilic silicone elastomers according to the present invention. FIG. 6 shows a preferred polyurethane which exhibits significant hydrophilicity when reacted with a polyorganosiloxane polymer according to the present invention. In the figures, n is generally 1-100. In FIG. 6, n is 1-100 (1-15 preferred) (1 preferred); x is 1-100 (15-25 preferred); y is 1-100 (3-15 preferred); and z is 1-25 (1-10 preferred) (1 preferred).

### SUMMARY OF THE INVENTION

The present invention relates to the use of a hydrophilic silicone elastomer as otherwise defined herein alone as a composition which exhibits emulsifier and other physicochemical characteristics, in certain preferred embodiments, in combination with an oil and optionally water, plus additional optional components to produce emulsions which are included in personal care products according to the present invention.

Hydrophilic silicone elastomers according to the present invention comprise a polyorganosiloxane polymer, which is often and preferably crosslinked and which contains appreciable quantities of hydroxyl groups or other moieties which instill hydrophilicity to produce a material that is primarily hydrophobic in character, but is sufficiently hydrophilic in order to be compatible with water and other polar solvents. In certain aspects, the silicone elastomer material is crosslinked with an allyl alcohol ethoxylate and/or a polyurethane polymer as otherwise described herein to provide hydrophilic character to the polyorganosiloxane polymer. In alternative embodiments, the polyorganosiloxane may be crosslinked or reacted with a second polyorganosiloxane polymer which may be further reacted with an allyl alcohol ethoxylate and/or a polyurethane polymer as otherwise described herein to produce hydrophilic silicone elastomers according to the present invention. The allyl alcohol ethoxylate and/or polyurethane reactant, in order to enhance solubility with the polyorganosiloxane reactant during formation of the hydrophilic silicone elastomer may itself be modified with a polyorganosiloxane in order to enhance solubility of the reactants during formation of the final hydrophilic silicone elastomer.

The hydrophilic silicone elastomer according to the present invention may vary significantly in chemical composition as described in greater detail hereinbelow.

The hydrophilic elastomers according to the present invention may be used alone as a composition which exhibits emulsifier and other physicochemical characteristics in personal care product compositions, or in certain alternative preferred embodiments, may be combined with an oil and optionally water, plus additional optional components to produce emulsions, especially water-in-oil and water-in-oil in water emulsions, which are included in personal care products according to the present invention. An emulsion according to the present invention comprises an effective amount of a hydrophilic silicone elastomer as otherwise described herein in combination with an oil, water and optionally, a secondary emulsifier (a traditional emulsifier known in the art).

Personal care product compositions according to the present invention comprise about 0.05% to about 50%, about 0.1% to about 20-25%, about 0.1% to about 15%, about 0.25% to about 10% by weight of a hydrophilic silicone elastomer composition according to the present invention, and the remaining portion of the personal care product composition comprising at least one additional component selected from the group consisting of an aqueous solvent (e.g. alcohol or other compatible solvent), a non-aqueous solvent, emollients, humectants, oils (polar and non-polar) conditioning agents, surfactants, thickeners/thickening agents, stiffening agents, emulsifiers, including secondary emulsifiers, medicaments, fragrances, preservatives, deodorant components, anti-perspirant compounds, skin protecting agents, pigments, dyes, coloring agents, sunscreens and mixtures thereof, among others

The present hydrophilic silicone elastomers may be used to form emulsions in combination with effective amounts of an oil and water and optionally a secondary (traditional) emulsifier. In the case of emulsion compositions, emulsions according to the present invention comprise about 5% to about 99.9% by weight of an oil, about 0% to about 94.9% by weight of water in combination with a hydrophilic silicone elastomer as otherwise described herein in an amount ranging from about 0.1% to about 25% by weight of the final emulsion composition, optionally in combination with additional components as described herein. Emulsion compositions comprise about 5% to about 94.9% by weight of an oil, about 5% to about 94.9% by weight of water in combination with about 0.1% to about 25% by weight of hydrophilic silicone elastomer as otherwise described herein. A secondary (traditional) emulsifier is optionally added to the emulsifier in effective amounts within the range of about 0.05% to about 15+%, about 0.1% to about 12.5%, about 0.5% to about 10% by weight of the emulsion composition. Emulsion compositions, after formulation, comprise about 1% to about 99%, about 2.5% to about 90%, about 5% to about 75%, about 7.5% to about 65%, about 10% to about 50% by weight of a personal care composition to which the emulsion is added, additional components including effective amounts of an aqueous solvent (e.g. alcohol or other compatible solvent), a non-aqueous solvent, emollients, humectants, oils (especially secondary oils which may be polar and non-polar), conditioning agents, surfactants, thickeners/thickening agents, stiffening agents, emulsifiers, including secondary emulsifiers, medicaments, fragrances, preservatives, deodorant components, anti-perspirant compounds, skin protecting agents, pigments, dyes, coloring agents, sunscreens and mixtures thereof.

### DETAILED DESCRIPTION OF THE INVENTION

The following terms are used to describe the present invention. In instances where a term is not specifically defined herein, the term shall be given its meaning as understood by those of ordinary skill in the art.

The term “patient or subject” is used to describe a mammal, including a human to which compositions according to the present invention may be applied.

The term “effective” is used, in context, to describe an amount or concentration of a compound, composition or component, as otherwise described herein which is included or used to provide an intended effect.

The term “personal care product” is used to describe a chemical composition used for the purpose of cleansing, conditioning, grooming, beautifying, or otherwise enhancing the appearance of the human body, especially keratinous tissue, including skin, nails and hair. Personal care products include skin care products, cosmetic products, antiperspirants, deodorants, perfume, toiletries, soaps, bath oils, feminine care products, hair-care products, oral hygiene products, depilatories, including shampoos, conditioners, hair straightening products and other hair care products, color cosmetics such as lipstick, creams, make-up, skin creams, lotions (preferably comprised of water-in-oil or oil-in-water emulsions), shave creams and gels, after-shave lotions and shave-conditioning compositions and sunscreen products, among numerous others.

Personal care products according to the present invention comprise an admixture of a hydrophilic silicone elastomer as otherwise described herein alone or in combination with an oil and water (emulsion) in the weight percentages as otherwise disclosed herein and at least one or more additional components selected from the group consisting of an aqueous solvent (e.g. alcohol or other compatible solvent), a non-aqueous solvent, emollients, humectants, oils (polar and non-polar) conditioning agents, surfactants, thickeners/thickening agents, stiffening agents, emulsifiers, including secondary emulsifiers, medicaments, fragrances, preservatives, deodorant components, anti-perspirant compounds, skin protecting agents, pigments, dyes, coloring agents, sunscreens and mixtures thereof, among others.

Preferred personal care products according to the present invention comprise about 0.01% to about 95% by weight of an emulsion which comprises a hydrophilic silicone elastomer as otherwise described herein, an oil and water, with the remainder of the composition comprising at least one additional component selected from the group consisting of an aqueous solvent (e.g. alcohol or other water compatible solvent), a non-aqueous solvent, emollients, humectants, a secondary oils (polar and non-polar), conditioning agents, emulsifiers, including secondary emulsifiers, surfactants, thickeners, stiffening agents, medicaments, fragrances, preservatives, deodorant components, anti-perspirant compounds, skin protecting agents, pigments, sunscreens and mixtures thereof, among others.

The term “hydrophilic silicone elastomer” describes a polyorganosiloxane polymer, which is often and preferably crosslinked and which contains appreciable quantities of hydroxyl groups or other moieties which instill hydrophilicity to produce a material that is primarily hydrophobic in character, but is sufficiently hydrophilic in order to be compatible with water and other polar solvents. In certain aspects, especially where the silicone elastomer is resembles rubber (resumes its original shape when a deforming force is removed), the material is crosslinked. The polyorganosiloxane polymer (silicone elastomer) according to the present invention may vary significantly in chemical composition but preferably is a polymeric composition comprised of

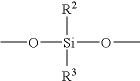

units, where R² and R³ are independently H, OH, C₁-C₁₀ (preferably C₁-C₃) alkyl, C₂-C₁₀ alkene (preferably vinyl or allyl) or aryl groups and may vary in average molecular weight Mₙ from about 1,000 to about 1,500,000 or more, preferably about 1,000 to about 100,000, more preferably about 2,500 to about 25,000 or more, depending upon the final viscosity and other characteristics desired. Silicone elastomers according to the present invention preferably contain a number of groups which are capable of crosslinking with an allyl alcohol ethoxylate according to the chemical structure:

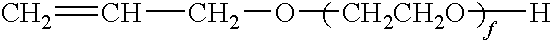

Where f is a positive integer from 1 to 20, 2 to 15, 3 to 15, 4 to 15, 5 to 15, 7 to 12, 8, 9, 10 or 11 (preferably 10); or
a functional group on a polyurethane polymer such as H, OH, NH₂, or an olefinic or other alkene group. The resulting polymer which incorporates the allyl alcohol ethoxylate and/or a polyurethane polymer is referred to as hydrophilic silicone elastomer. In other aspects of the invention, the silicone elastomer may be crosslinked with another polysiloxane polymer to the exclusion of, or in addition to the allyl alcohol ethoxylate and/or polyurethane polymer. In still further embodiments, allyl alcohol ethoxylate and/or polyurethane polymer may first be reacted with an organopolysiloxane compound and then reacted with the polyorganosiloxane compound in order to enhance the solubility characteristics of reactions to provide a final hydrophilic silicone polymer.

In preferred aspects, the allyl alcohol ethoxylate comprises about 1% to about 15% by weight of the monomers/polymers which ultimately form certain embodiments of the hydrophilic silicone elastomer according to the present invention. In aspects where a polyurethane polymer is complexed or crosslinked with the polyorganosiloxane polymer to provide final hydrophilic silicone elastomers (in contrast to polyurethane-silicone reactants), the polyurethane polymer comprises about 1% to 50% or more (e.g., up to about 75%), about 2% to about 45%, about 5% to about 25% by weight of the final polymeric composition in order to provide a hydrophilic component. In instances where the polyurethane is first reacted with a polyorganosiloxane to produce a polyurethane-silicone reactant which is further reacted with a polyorganosiloxane to produce a final hydrophilic silicone elastomer, the polyurethane polymer comprises about 50% to about 99%, about 60% to about 99%, about 75% to about 98%, about 80% to about 95% of a mixture of polyurethane and polyorganosiloxane to produce polyurethane-polyorganosiloxane reactants.

The hydrophilic silicone elastomers according to the present invention are preferably crosslinked (the reaction preferably occurring between two olefinic groups on the molecules to be crosslinked), with another polymeric silicone compound or preferably, with a polyurethane compound, which itself is optionally reacted or crosslinked with a polyorganosiloxane (which preferably instills hydrophilic character to the final polymeric composition either alone or in combination with the allyl alcohol ethoxylate residues which are incorporated into the polymer) as otherwise described here. For example, with silicone elastomers which contain an Si—H group, reaction with a free hydroxyl group on a polyurethane composition produces a crosslinked Si—O-polyurethane polymer group. The Si—H group (as well as any pendant alkenyl group) may also be used to introduce the allyl alcohol ethoxylate monomer into the silicone polymer. In the case of a Si—OH group or Si—NH₂ group, reaction with an electrophilic group on a polyurethane compound such as an isocyanate, ester group (or activated ester) or other electrophilic group will result in a crosslinked silicone elastomer-polyurethane composition. In the case of a Si-alkene group, reaction with an olefinic group or other activated group on the polyurethane (through dimethylsilicone hydride groups that are contained on a separate crosslinking silicone polymer) can produce a silicone elastomer-polyurethane polymer crosslinked through the double bonds on each of the elastomer and polyurethane. These groups can also be used to introduce allyl alcohol ethoxylate groups and/or polyurethane compounds having the appropriate pendant group. Alternatively, the hydrophilic silicone elastomer (hydrophilic through introduction of allyl alcohol ethoxylate groups) and polyurethane may simply be admixed without further crosslinking/polymerization.

In certain preferred aspects of the present invention, the final hydrophilic silicone elastomer is prepared from a reaction mixture which comprises a silicone elastomer as described above (which may optionally comprise an allyl alcohol ethoxylate as otherwise described herein) and a reactive polyurethane wherein the elastomer and polyurethane are covalently linked (crosslinked or uncrosslinked, but covalently bonded). Thus, hydrophilic silicone elastomers may comprise the reaction product of a silicone elastomer as otherwise described herein (preferably containing a number of hydrosilicon groups and optionally and preferably, at least one alkenyl group) in combination with about 1% to about 15% by weight, preferably, about 5% to about 10% by weight of an allyl alcohol ethoxylate as otherwise described herein (based upon the silicone elastomer, allyl alcohol ethoxylate and any optional reactants included in the reaction mixture). Alternatively, hydrophilic silicone elastomers comprise the reaction product of a silicone elastomer as otherwise described herein (including silicone elastomers which have been prepared using allyl alcohol ethoxylates as described above) in combination with a polyurethane compound (which may be optionally modified with a polyorganosiloxane compound for solubility purposes during the reaction), optionally in combination with an allyl alcohol ethoxylate (the allyl alcohol ethoxylate in such case may range from about 1% to about 15%, about 1% to about 10%, about 1% to about 7.5%, about 1% to about 5% of the reaction mixture which provides the hydrophilic silicone elastomer). For preparation of hydrophilic silicone elastomers which contain a bonded polyurethane to optionally instill at least a portion of the hydrophilic character to the final silicone elastomer, the silicone elastomer precursor comprises about 5% to about 95% by weight of the reaction mixture, about 10% to about 90%, about 15% to about 85%, about 20% to about 80%, about 25% to about 75%, about 30% to about 70% about 35% to about 65%, about 40% to about 60%, about 45% to about 55% and about 50% of the reaction mixture and the polyurethane compound comprises about 5% to about 95% by weight of the reaction mixture, about 10% to about 90%, about 15% to about 85%, about 20% to about 80%, about 25% to about 75%, about 30% to about 70% about 35% to about 65%, about 40% to about 60%, about 45% to about 55% and about 50% of the reaction mixture. As noted above, when allyl alcohol ethoxylate is an additional reaction component, the allyl alcohol ethoxylate comprises about 1% to about 15% as described above of the reaction mixture.

In certain preferred embodiments, the polyorganosiloxane polymers (silicone elastomers) which are used to prepare hydrophilic silicone elastomers (by reaction with an allyl alcohol ethoxylate and/or a polyurethane compound as otherwise described herein) according to the present invention have the following structure:

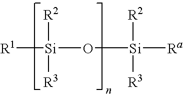

Where R¹ and R^{a} are independently H, an optionally substituted C₁-C₆ alkyl group (substitution with OH or a C₁-C₃ alkyl group which itself may be optionally substituted with a hydroxyl group) or an optionally substituted C₂-C₆ alkenyl group (which term may include an acrylate or methacrylate group);

Each R² and R³ is independently H, OH, or a C₁-C₃ alkyl group; and

n is from 5 to 50,000, about 10 to about 25,000, about 100 to about 10,000.

Additional preferred polyorganosiloxane polymers (silicone elastomer compounds) which may be used to prepare hydrophilic silicone elastomers (in combination with allyl alcohol ethoxylate and/or polyurethane compounds), according to the present invention have the following structure:

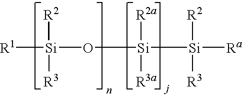

Where R¹ and R^{a} are independently H, an optionally substituted C₁-C₆ alkyl group (substitution with OH or a C₁-C₃ alkyl group which itself may be optionally substituted with a hydroxyl group) or an optionally substituted C₂-C₆ alkenyl group (which term may include an acrylate or methacrylate group);

Each R² and R³ is independently H, OH, or a C₁-C₃ alkyl group (preferably a C₁-C₃ alkyl group, preferably a methyl group), preferably R² and R³ are both C₁-C₃ alkyl groups, preferably both are the same C₁-C₃ alkyl group, preferably methyl groups;

Each R^{2a} and R^{3a} is independently H, OH, or a C₁-C₃ alkyl group, preferably at least one of R^{2a} or R^{3a} is H and the other is a C₁-C₃ alkyl group;

n is from 5 to 50,000, about 10 to about 25,000, about 100 to about 10,000, about 100 to 5,000; about 500 to 5,000; about 500 to about 2,500; about 100 to about 1,000, about 150 to about 1,000; and

j is from 0 to 50, preferably 1 to about 25, about 1 to 4, about 2 to 15 about 3 to 10; about 5 to 10.

Other preferred polyorganosiloxane polymers (silicone elastomers) for use in the present invention include polyorganosiloxane polymers which are crosslinked with a polysiloxane (e.g., end-capped with reactive Si—H groups which react with double bonds on the uncrosslinked polysiloxane polymer) crosslinking agent. These complex polyorganosiloxane polymers may be further reacted with allyl alcohol ethoxylate groups and/or polyurethane groups (each of which groups may be reacted with a polyorganosiloxane before further reaction with a polyorganosiloxane) which instill hydrophilic character to the final silicone elastomer composition. Silicone elastomers generally range in size from an average molecular weight of at least about 500 to upwards of several million or more, more preferably about 2000 to about 1,000,000, more preferably about 5,000 to about 500,000. Silicone elastomers are generally formed by reacting a polysiloxane polymer which contains at least one side chain comprising a hydrocarbon which contains at least one reactive double bond (e.g. a vinyl group or an allyl group) which is reactive with an Si—H bond. The crosslinker may vary in size, but generally ranges in size from a molecular weight of about 125 to several thousand or more, with a preferred molecular weight range of about 200 to about 750.

Preferred silicone elastomers which may be modified to hydrophilic silicone elastomers for use in the present invention include for example, the silicone elastomers which are disclosed in U.S. Pat. No. 6,936,686, which is incorporated by reference in its entirety herein.

The term “polyurethane” shall mean, within the context of its use, a polymeric urethane compound comprising at least one and preferably, two or more urethane linkages which are generally formed by reacting at least one compound containing a free alcohol (primary, secondary or tertiary), preferably at least one compound containing at least two alcohol groups (“polyol”) and a diisocyanate compound. Thus, the term polyurethane as used herein incorporates dimer urethanes (those compounds which contain a single urethane bond) which are formed from a monohydric alcohol of varying structure, which structure may contain, for example, an active group or a protected active group such as a silyl-protected hydroxyl group or amine group wherein the protecting may be removed subsequent to formation of the polyurethane or an olefinic group (such as for example, a vinyl group, acrylate or methacrylate group) which can participate in a reaction with a group from the silicone elastomer to produce a crosslinked silicone elastomer/polyurethane composition. In addition, polyurethanes according to the present invention preferably are formed by reacting at least one polyol (a compound which is either hydrocarbon or siloxane based and which contains at least two free alcohol groups) with a diisocyanate to produce a polyurethane, with the polyol optionally and preferably containing at least one functional group which does not participate in the polymerization reaction to form the polyurethane composition, but which, subsequent to the polymerization reaction, can be used to crosslink the polyurethane composition to a silicone elastomer in preferred compositions according to the present invention. In preferred aspects of the invention, polyurethane compounds which are reacted with a silicone elastomer to produce hydrophilic silicone elastomers preferably have sufficient hydrophilic character (for example, by containing sufficient hydroxyl groups and/or ethoxylated-polyethylene oxide or peg groups) to instill hydrophilic character to the final hydrophilic silicone elastomers according to the present invention.

Preferred urethane polymers according to the present invention have the general structure:

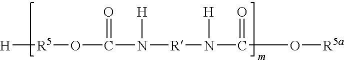

Where R⁵ is an optionally substituted hydrocarbon or optionally substituted siloxane group, preferably, an optionally substituted (with hydroxyl groups and/or peg groups comprising from 1 to 100 or 2 to 25 ethylene oxide units) C₁-C₅₀ hydrocarbon group containing at least one olefinic group or a polyethylene oxide group comprising between 1 and 500, 2 and 100, 5 and 25, 5 and 20, 5 and 15 ethylene oxide groups which may be optionally endcapped with or contain a polymerizable group such as an alkenyl or (meth)acrylate group, or preferably a siloxane group according to the structure:

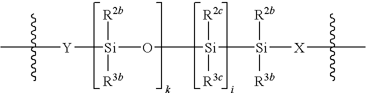

and R^{5a} is an optionally substituted hydrocarbon (which may containing hydroxyl and/or peg groups as otherwise described here) or a siloxane group, preferably, an optionally substituted C₁-C₅₀ hydrocarbon group, optionally containing at least one olefinic group, or a siloxane group according to the structure:

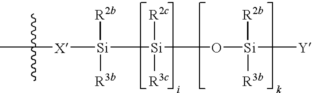

Wherein Y is absent, O or a —W—(O—Z)ᵣ-Q-(CH₂)_{q}-T-group;
X is absent or a -TCH₂)_{q}-Q-(Z—O)ᵣ—W′-group;
X′ is absent or a —W′—(O—Z)ᵣ-Q-(CH₂)_{q}-T-group;
Y′ is absent or a -TCH₂)_{q}-Q-(Z—O)ᵣ—W² group;
W is absent when r is an integer of 1 or more and W is absent or O when r is 0;
Q is absent or O;
q is an integer from 0 to 10, preferably 1 to 6, preferably 1 to 3;
r is an integer from 0 to 100, 0 to 40, preferably 1 to 20 or 1 to 10, with the proviso that q or r is at least 1;
T is absent or O;
W′ is absent when r is 0 and is a Z group when r is 1 or more;

### W² is H;

Z is independently an ethylene group, a propylene group or a mixture of ethylene and propylene groups;
R^{2b} and R^{3b} are each independently H or a C₁-C₃ alkyl group (preferably a C₁-C₃ alkyl group, preferably a methyl group), preferably R² and R³ are both C₁-C₃ alkyl groups, preferably both are the same C₁-C₃ alkyl group, preferably methyl groups;
R^{2c} and R^{3c} are independently selected from H, an optionally substituted C₁-C₆ alkyl group (substitution with OH or a C₁-C₃ alkyl group which itself may be optionally substituted with a hydroxyl group) or an optionally substituted C₂-C₆ alkenyl group (which alkenyl may include an acrylate or methacrylate group);
R′ is an optionally substituted C₂ through C₃₆ (preferably, C₆ through C₂₂, most preferably an isophorone group) linear, cyclic or branch-chained saturated or unsaturated hydrocarbon group (which may be monomeric or dimeric, an aromatic group, including a phenyl or benzyl group or substituted phenyl or benzyl group, an alkylphenyl, alkylbenzyl or substituted alkylphenyl or alkylbenzyl group);
i is an integer from 0 to 50, preferably 0, 1, 2, 3, 4, 5, 6, 7, 8, 9 or 10 (preferably 0 or 1);
k is an integer from 0 to 100, 1 to 100, about 5 to 50, about 10 to 45, preferably about 20 to 40; and
m is from 1 to 100,000, about 1 to 25,000, about 5 to 25,000, about 50 to 20,000, about 50 to 20,000, about 100 to 20,000, about 100 to 10,000, about 200 to 5,000, about 250 to 2,500, about 500 to about 2,000, 1 to about 1,000, 1 to about 750, 2 to about 650, about 50 to 15,000, about 10 to 10,000, about 200 to 5,000, about 250 to about 2,500, about 5 to about 150, about 3 to 100, about 5 to 250. Preferably, the polyurethane according to the present invention is obtained by reacting a polyol (which may be hydrocarbon based or siloxane based and contains at least two hydroxyl groups) with a diisocyanate compound to produce a polyurethane composition accordingly.

In certain preferred aspects, R⁵ is a O—R⁶ group and R^{5a} is a R^{6a}—OH group where R⁶ and R^{6a} are each independently a optionally substituted hydrocarbon or an optionally substituted siloxane group as set forth for R⁵ and R^{5a}, respectively and generally described above.

One or more polyols and/or diisocyanates may be used to produce polyurethane polymers according to the present invention, with preferred polyols having, in addition to at least two free alcohol groups to participate in polymerization reactions to form polyurethanes, at least one additional reactive functional group which can serve to crosslink silicone elastomers in the present invention, and with the diisocyanate preferably being isophorone diisocyanate. Further preferred polyols contain multiple hydroxyl groups or alternatively, polyethylene oxide groups wherein the peg groups contain from 2 to 100 ethylene oxide groups, preferably 3 to 50, 5 to 25 or 5 to 10.

Alternative polyurethanes according to the present invention also are prepared from a diisocyanate, preferably isophorone diisocyanate and castor oil, glycerin and glycerin esters, propylene glycol and its esters, dipropylene glycol and its esters, alkyl amines, ethoxylated alkyl amines, propoxylated alkyl amines, silicone ethoxylates and silicone propoxylates, among others.

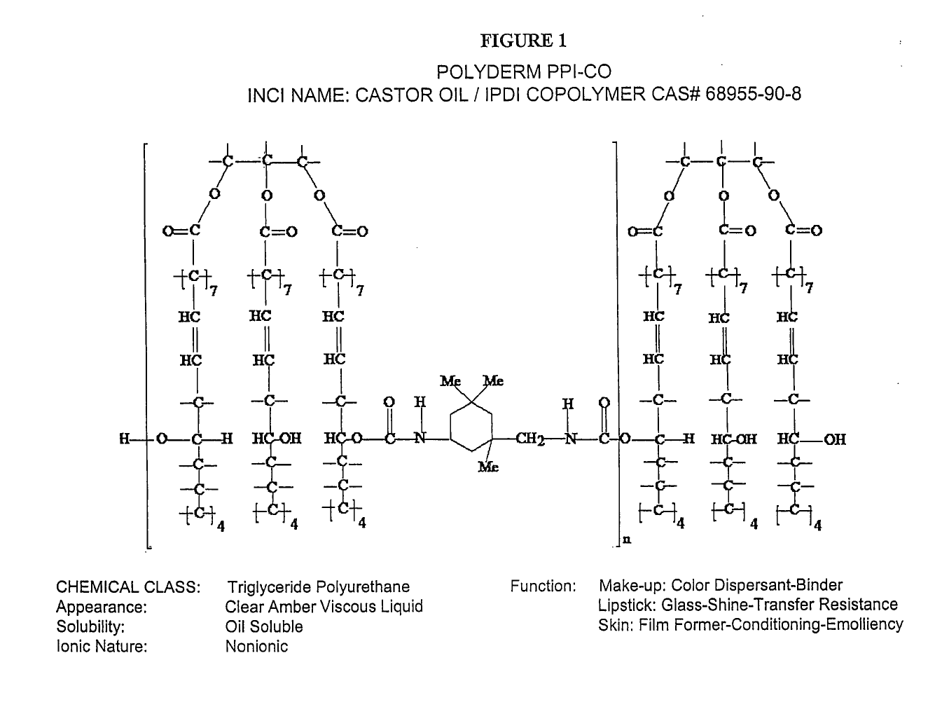

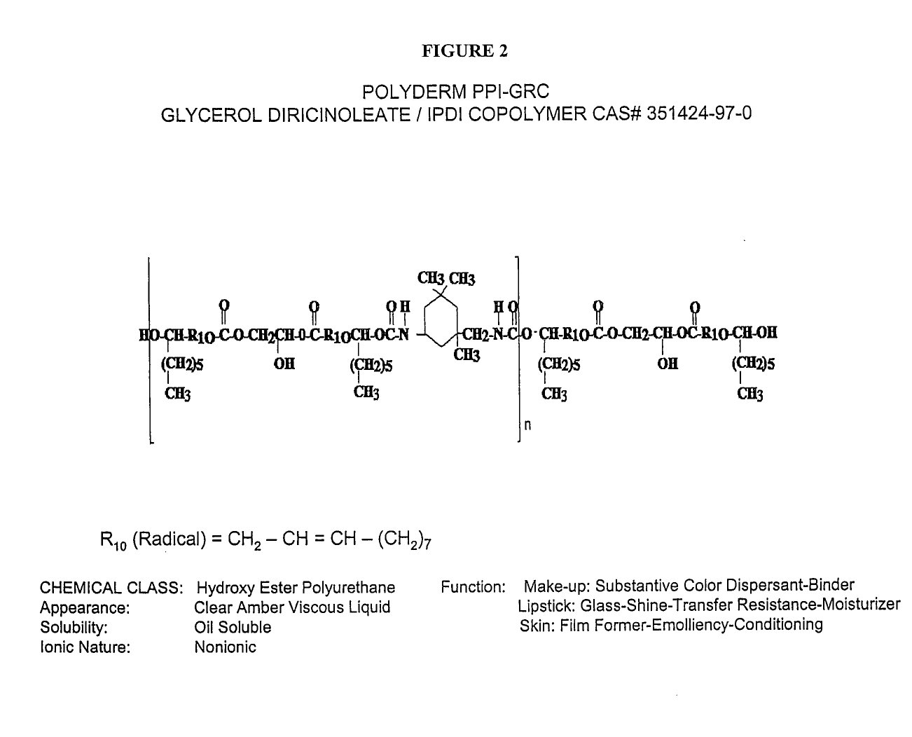

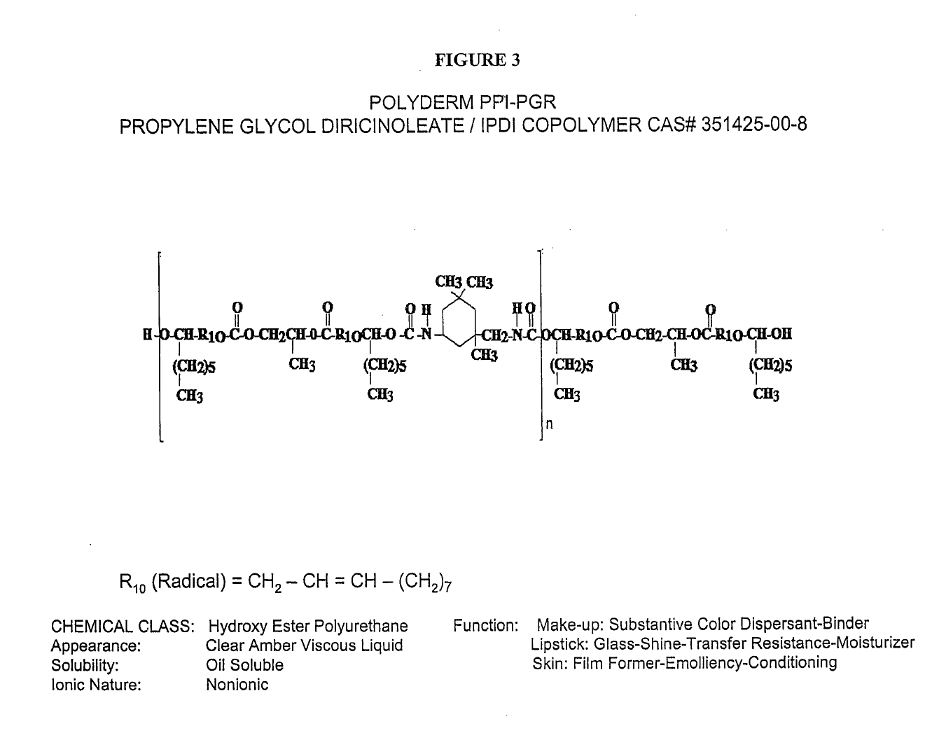

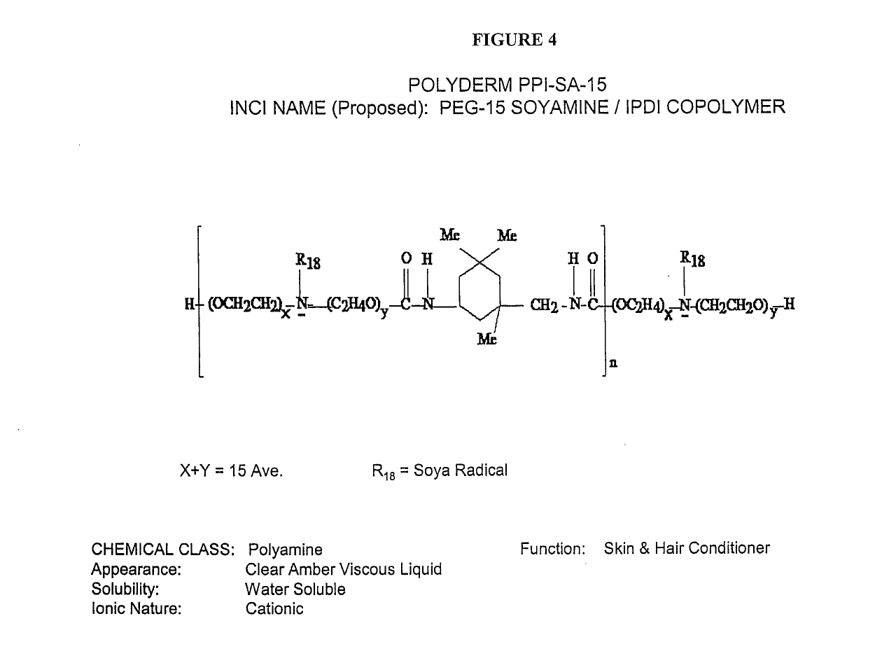

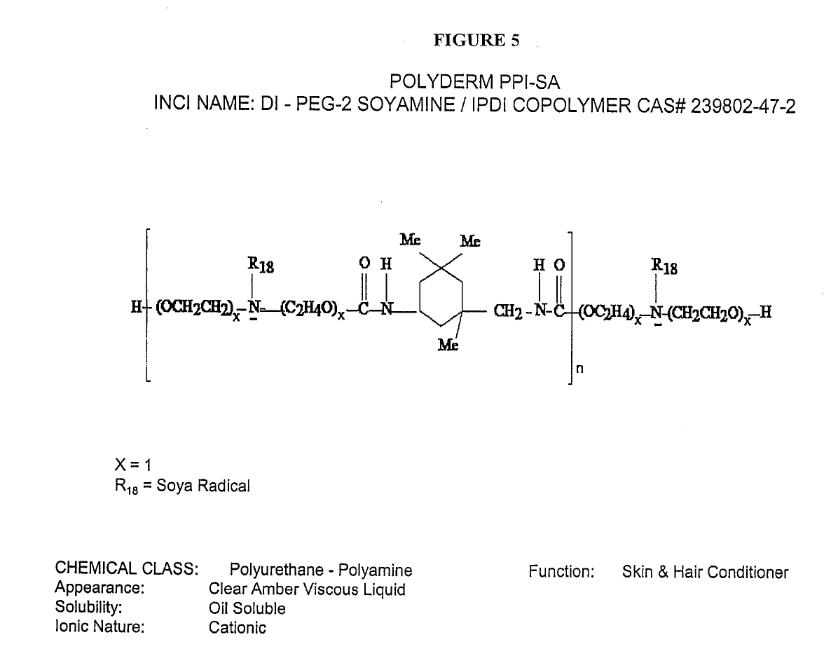

Certain polyurethane compositions for use in the present invention include the various polyurethane compositions which are disclosed in FIGS. 1-8 (includes 8A and 8B) hereof, which are available commercially from Alzo International, Inc. of Sayreville, N.J., USA.

The term “polyol” refers to a hydrocarbon or siloxane based compound having at least two free hydroxyl groups which can participate in a reaction with diisocyanate to provide a polyurethane composition. In preferred aspects of the invention, a polyol according to the present invention, in addition to the two free hydroxyl groups which react with diisocyanate compounds, also contains an additional “reactive functional group” which, subsequent to the formation of the polyurethane compound, may participate in a crosslinking reaction with a reactive functional group on a silicone elastomer admixed with the polyurethane, to produce crosslinked silicone elastomer/polyurethane compositions The term “monohydric alcohol” refers to a compound containing a single hydroxyl group which may react with a diisocyanate compound to produce dimer urethane compounds according to the present invention. Monohydric alcohols advantageously contain at least one reactive functional group which, after formation of the dimer urethane, can react with a reactive group on a silicone elastomer admixed with the dimer urethane to produce a crosslinked silicone elastomer/polyurethane compositions.

The polyol(s) used to polymerize with diisocyanate may vary widely in character from hydrophilic (polar) to hydrophobic non-polar, but are preferably hydrophilic in nature. Although a large number of polyols can be used to produce polyurethane compositions according to the present invention, preferred polyols include triglycerides which contain fatty acids having free hydroxyl groups and/or olefinic groups such as castor oil triglycerides or other triglycerides, glycerol, substituted glycerols or polyglycerols such as C₁₀-C₂₄ di-fatty polyglycerol (preferably, polyglycerol-2-diisostearate), di-fatty alkanolmonoglycerol, such as glycerol diricinoleate, polyethylene glycol alkylamines, especially polyethyleneglycol fatty amines, such as PEG-15 cocamine, or di-PEG-15 soyamine or related dipolyethylene glycol fatty amines, including di-PEG soyamine, polyethyleneglycol, substituted polyethyleneglycol, such as propyleneglycol diricinoleate, polydialkylsiloxane such as polydimethylsiloxane (e.g. dimethicone), or a di-polyethyleneglycol dimethicone, or related polysiloxane. Polyols are polymerized with a diisocyanate compound, preferably isophorone diisocyanate.

The term “diisocyanate” is used throughout the specification to describe a linear, cyclic or branch-chained hydrocarbon having two free isocyanate groups. The term “diisocyanate” also includes halogen substituted linear, cyclic or branch-chained hydrocarbons having two free isocyanate groups. Exemplary diisocyanates include, for example, isophoronediisocyanate, m-phenylene-diisocyanate, p-phenylenediisocyanate, 4,4-butyl-m-phenylene-diisocyanate, 4-methoxy-m-phenylenediisocyanate, 4-phenoxy-m-phenylenediisocyanate, 4-chloro-m-phenyldiisocyanate, toluene diisocyanate, m-xylylene diisocyanate, p-xylylene diisocyanate, 1,4-napthalene diisocyanate, cumene-1,4-diisocyanate, durene diisocyanate, 1,5-napthylene diisocyanate, 1,8-napthylene diisocyanate, 1,5-tetrahydronapthylene diisocyanate, 2,6-napthylene diisocyanate, 1,5-tetrahydronapthylene diisocyanate; p,p-diphylene diisocyanate; 2,4-diphenylhexane-1,6-diisocyanate; methylene diisocyanate; ethylene diisocyanate; trimethylene diisocyanate, tetramethylene diisocyanate, pentamethylene diisocyanate, hexamethylene diisocyanate, nonamethylene diisocyanate, decamethylene diisocyanate, 3-chloro-trimethylene diisocyanate and 2,3-dimethyltetramethylene diisocyanate, among numerous others. Isophorone diisocyanate is the preferred diisocyanate used in the present invention.

Polyurethane compounds used in the present invention are prepared by well known methods in the art. A general scheme involves reaction of at least about two moles of a monohydric alcohol or at least about one mole of (purchased commercially or prepared using well known methods in the art) polyol with at least one mole of a diisocyanate in the presence of heat and either an amine or tin catalyst such as stannous octanoate. Each isocyanate moiety is thereby converted to a urethane moiety while maintaining the presence of “free and unreacted” reactive functional groups which may be later used to crosslink the silicone elastomer in admixture. Heat is maintained until a suitable acid number is attained and the reaction mass is cooled, washed, neutralized dried and distilled if necessary. The product should be essentially odorless, essentially water-white in color and contain a substantial absence of free fatty acid or residual catalyst.

The polyurethane and polyorganosiloxane (silicone elastomer) are reacted together to produce a hydrophilic silicone elastomer composition according to the present invention. Preferably, a silicone elastomer containing at least one functional reactive group (e.g., Si—H, Si—OH, NH₂, Si—C═C group) is admixed with a polyurethane also containing at least one reactive functional group (OH, NH₂, olefin (vinyl) or acrylate or methacrylate group and then reacted to provide a crosslinked silicone elastomer/polyurethane polymeric material, having varied viscosity, but preferably being presented as a gel. The silicone elastomer and polyurethane are reacted preferably without solvent or in the presence of solvent according to the nature of the reactive functional group on the silicone elastomer and the polyurethane and the type of reaction which produces a crosslinked product, where applicable. The reactions may be conducted at temperatures at (ambient) or below room temperature, but preferably the reactions occur at elevated temperatures alone or in the presence of a catalyst or acid scavenger (such as triethyl amine, etc.). The resulting crosslinked silicone elastomer/polyurethane polymer (especially the final viscosity) may vary according to the reaction conditions chosen to effect the crosslinking reaction.

The silicone elastomer/polyurethane admixture/polymer prepared above may be added to a number of components to product favorable characteristics in personal care products, including skin care products, cosmetic products, antiperspirants, deodorants, perfume, toiletries, soaps, bath oils, feminine care products, hair-care products, oral hygiene products, depilatories, including shampoos, conditioners, hair straightening products and other haircare products, color cosmetics such as lipstick, creams, make-up, skin creams, lotions and sunscreen products, among numerous others.

Compounds of the present invention may be used as emulsifiers having a number of additional characteristics including emollient characteristics for the skin and epithelial tissue such as hair, ungual tissue (nails), skin and related mucous membranes, especially given the combined attributes of emolliency (from the silicone elastomer) and skin adherence, viscosity enhancement and favorable skin interaction (generally) and wettability and other attributes (which can be formulated into the polymer depending upon which polyurethane is chosen). By addition of an effective amount of the present compositions, emulsion formulations which may be included in personal care products, including cosmetic and toiletry products will acquire a soothing and favorable interaction which promotes skin adherence, moisturization, wettability and favorable viscosity attributes of the final personal care formulation. In addition, because the size of the silicone elastomer and polyurethane can be varied substantially, numerous characteristics may be “dialed in” to the final hydrophilic silicone elastomers in addition to the basic emulsifier characteristics and incorporated into personal care products ranging from lotions and creams to thickened formulations to be used in stick deodorants and related products can be readily formulated.

Effective amounts of the present compositions may also serve a dual function, for example, as emulsifiers exhibiting gloss-producing characteristics for lipsticks and lip balm formulations in the personal care, cosmetic and toiletry industries as a substitute(s) for castor oil normally used in such formulations, especially where the polyurethane is made from castor oil. The compounds of the present invention exhibit outstanding solubility characteristics for producing water-in-oil or oil-in-water emulsions and may form the basis for numerous and varied personal care compositions.

Hydrophilic silicone elastomers according to the present invention exhibit one or more of a number of unexpected characteristics including providing compositions containing polyurethanes which do not exhibit a typical “sticky tactile” sensation when deposited on the skin of a subject (such as an animal, including a human) and provide a smooth, non-tacky feel which is especially advantageous for bodycare lotions and other personal care compositions used on the skin and hair of a subject. In addition, the compositions of the present invention provide “substantivity” to personal care products and can be used to accommodate functional ingredients, especially including hydrophilic functional ingredients such as polar hydrophilic materials. Because of the hydrophilic nature of the compositions, it is easier to formulate water-in-oil emulsions, including water-in-oil in water emulsions, which results in an emulsion or final personal care composition which accommodates large amounts of water, thus reducing the cost of components and the final cost of the formulated personal care composition.

In addition, hydrophilic silicone elastomer compositions according to the present invention may be used advantageously as couplers (in emulsions or in compositions which are not emulsions)—for example, to couple a hydrophilic components such as water and an aliphatic component (such as an oil, fatty waxes and esters) into a single formulation. This is based upon the tendency of certain compositions according to the present invention to sort into distinct areas of hydrophilicity and hydrophobicity which can accommodate hydrophilic and hydrophobic components of varying physicochemical characteristics, thus facilitating the compatibility of disparate physicochemical entities in a single compound.

The term “effective amount” is used throughout the present specification to describe concentrations or amounts of compounds according to the present invention which, when used in context, are effective in conveying desired traits such as emulsification (emulsifiers), emolliency, wettability, skin adherence, storage stability, and/or solubility to a formulation of a personal care product or are used to produce a compound or composition according to the present invention.

The term “unsubstituted” is used to describe a hydrocarbon moiety such as an alkyl group or alkene or related unsaturated group which contains only hydrogen atoms bonded to carbons within the moiety. It can include aryl (aromatic groups such as substituted phenyl) groups, as well. The term “substituted” is used to describe a hydrocarbon moiety which contains, within the context of its use, a pendant hydroxyl group (in preferred aspects numerous alcohol groups, an ether group (such as within a glycol or polyglycol/peg, glycerol or polyglycerol or other group), a keto group, an amine (which may itself be substituted with alkyl groups, including fatty (C₈-C₃₀) alkyl groups or alkanol groups, for example), an alkyl or alkene group attached to a carbon atom of the moiety. The number of carbon atoms within a substituent group may vary from 0 to 30 or more, 0 to 24 or more, 0 to 18, 0 to 12, 0 to 10, 1 to 8, and 1 to 6 and may contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more carbon atoms, depending upon the context of the use of the compound to which the substituent is attached.

The term “oil” is used throughout the specification to describe any of various lubricious, hydrophobic and combustible substances obtained from animal, vegetable and mineral matter. These are used to form emulsions of the present invention. Emollient oils for use in the present invention may include petroleum-based oil derivatives such as purified petrolatum and mineral oil. Petroleum-derived oils include aliphatic or wax-based oils, aromatic or asphalt-based oils and mixed base oils and may include relatively polar and non-polar oils. “Non-polar” oils are generally oils such as petrolatum or mineral oil or its derivatives which are hydrocarbons and are more hydrophobic and lipophilic compared to synthetic oils, such as esters, which may be referred to as “polar” oils. It is understood that within the class of oils, the use of the terms “non-polar” and “polar” are relative within this very hydrophobic and lipophilic class, and all of the oils tend to be much more hydrophobic and lipophilic than the water phase which is used to produce the water-in-oil emulsion of the present invention. Preferred hydrophobic oils for use in the present invention include mineral oil and petrolatum. Preferred less hydrophobic (i.e., more polar) oils for use in the present invention include a number of maleates, neopentanoates, neopentanoyls, citrates and fumarates, and any other cosmetically acceptable ester emollient.

In addition to the above-described oils, certain essential oils derived from plants such as volatile liquids derived from flowers, stems and leaves and other parts of the plant which may include terpenoids and other natural products including triglycerides may also be considered oils for purposes of the present invention.

Petrolatum (mineral fat, petroleum jelly or mineral jelly) and mineral oil products for use in the present invention may be obtained from a variety of suppliers. These products may range widely in viscosity and other physical and chemical characteristics such as molecular weight and purity. Preferred petrolatum and mineral oil for use in the present invention are those which exhibit significant utility in cosmetic and pharmaceutical products. Cosmetic grade oils are preferred oils for use in the present invention.

Additional oils for use in the present invention may include, for example, mono-, di- and tri-glycerides which may be natural or synthetic (derived from esterification of glycerol and at least one organic acid, saturated or unsaturated, such as for example, such as butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others. In addition, a number of other oils may be used, including C₁₂ to C₃₀ (or higher) fatty esters (other than the glyceride esters, which are described above) or any other acceptable oil.

In general, hydrophilic silicone elastomer compositions according to the present invention are included in personal care products/formulations in effective amounts, i.e., amounts which produce an intended effect. The amount of composition generally ranges from about 0.01% to about 50% by weight or more of personal care formulations according to the present invention. Alternatively, compositions according to the present invention may be included in final personal care compositions in amounts ranging from about 0.05% to about 45% by weight, about 0.1% to about 40% by weight, about 0.25% to about 30% by weight, about 0.25% to about 20% by weight, about 0.5% to about 15% by weight, about 0.75% to about 10% by weight, about 1% to about 7.5% by weight, about 1% to about 5% by weight and about 1% to about 3% by weight of the final personal care composition.

In preferred embodiments of emulsion-based formulations (wherein the formulation comprises an oil, water and the present composition as an emulsifier, compositions according to the present invention are included in amounts ranging from about 0.1% to about 25% by weight, in addition to the oil and water and optionally, other components. Emulsions according to the present invention may be used in any number of personal care products, but find particularly useful applicability in formulations which are based upon lotions and/or skin creams.

The compositions according to the present invention may be used in numerous additional compositions. In the case of shampoos and conditioners, compositions according to the present invention are included in amounts ranging from about 0.1% to about 15% by weight of the formulation, in certain cases to instill conditioning attributes in addition to surfactant-like qualities. One can use amounts up to about 20% to 25% in shampoos and conditioners. For example, in haircare products, such as shampoos, rinses, conditioners, hair straighteners, hair colorants and permanent wave formulations, the compositions according to the present invention preferably comprise about 0.1% to about 20% by weight, more preferably about 0.25% to about 5% by weight of the final end-use hair-care composition. Other components which may be included in hair-care formulations include, for example, a solvent or diluent such as water and/or alcohol, other surfactants, emulsifiers, thickeners, coloring agents, dyes, preservatives, additional conditioning agents and humectants, among numerous others.

In the case of shave creams and gels, after-shave lotions and shave-conditioning compositions (for example, pre-electric shave formulations), the compositions according to the present invention are included in amounts ranging from about 0.25% to about 15% or more by weight, more preferably about 0.5% to about 10% by weight. Other components which may be included in these end-use compositions include, for example, water, and at least one or more of emollients, humectants and emulsifiers, thickeners and optionally, other conditioning agents, medicaments, fragrances and preservatives.

In the case of skin lotions and creams, the present compositions are included in amounts ranging from about 0.25% to about 15% by weight, more preferably, about 0.5 to about 10% by weight. Additional components which may be employed in these compositions include, for example, water, emollients and emulsifiers, surfactants, oils, and optionally, other conditioning agents, thickeners, medicaments, fragrances and preservatives.

In the case of sunscreens and skin-protective compositions, the present compositions are included in amounts ranging from about 0.25% to about 15% or more by weight, preferably about 0.5% to about 7.5% by weight of the final formulations. These compositions form the basis of lotions or skin creams which may be used to deliver pigments and/or sunscreen components in compositions according to the present invention. Additional components which may be employed in these compositions may include, for example, a UV absorbing composition such as para-amino benzoic acid (PABA) or a related UV absorber or a pigment such as TiO₂ and optional components including, for example, one or more of an oil, water, suspending agents, other conditioning agents and emollients, among others.

In the case of bar and liquid soaps, compositions according to the present invention are included for their surfactant and emollient-like qualities in amounts ranging from about 0.25% to about 20% by weight or more, preferably about 0.5% to about 10% by weight. Additional components which may be included in bar and liquid soaps include water and surfactants and optionally, bactericides, fragrances and colorants, among others.

Other personal care products, not specifically mentioned, generally comprise about 0.1% to about 50% by weight of a composition according to the present invention and other components of personal care products as otherwise set forth in detail herein.

The present invention also relates to a method of preparing a hydrophilic silicone elastomer according to the present invention by reacting a polyorganosiloxane as otherwise described herein with about 0.5% to about 25%, about 1% to about 15%, about 1% to about 10% by weight of the reaction mixture of an allyl alcohol ethoxylate monomer and/or a polyurethane as otherwise described either stepwise, or in a single step reaction, preferably in a single pot. A second polyorganosiloxane may be reacted with the first polyorganosiloxane in an amount ranging from about 0.5% to about 50%, about 1% to about 25%, about 1% to about 20% about 1% to about 15% by weight of a mixture of first and second polyorganosiloxanes before reaction with the allyl alcohol ethoxylate monomer and/or polyurethane. The reaction is optimally prepared in a single pot.

The following examples are intended to be illustrative of the invention concepts, and are meant to provide formulas and manufacturing methods to show some of the variations and applications that are possible. They are

| | Example 1 | Example 2 | Example 3 |
|---|---|---|---|
| Ingredients | % w/w | % w/w | % w/w |
| Dermol 99 (Isononyl Isononanoate) | 15.7 | xxx | xxx |
| Isododecane | xxx | 63.0 | 63.0 |
| Hydrophilically modified silicone | 1.0 | xxx | 4.0 |
| elastomer (A) | | | |
| Hydrophilically modified silicone | xxx | 4.0 | xxx |
| elastomer (B) | | | |
| Water | 66.7 | 20.0 | 20.0 |
| D5 Cyclomethicone | 16.4 | xxx | xxx |
| Methylisothiazolinone (1.5% aq. | 0.2 | xxx | xxx |
| solution) | | | |
| Glycerin | — | 13.0 | 13.0 |
| | 100.0 | 100.0 | 100.0 |

The silicone elastomers shown in these examples and all other examples throughout this description are made per U.S. Pat. No. 6,936,686 the entire contents of which are incorporated by reference herein, from a polyorganohydrosiloxane of about 3500 to 4000 molecular weight and with 6 to 7 pendant hydrosilane groups per molecule that is reacted with a 20,000 to 25,000 molecular weight vinyl terminated polydimethylsiloxane with an added 5 or 10 percent by weight of allyl alcohol 10 mole ethoxylate. Addition of the ethoxylated allyl alcohol gives the elastomer significant hydrophilicity. The hydrophilic crosslinked elastomer is initially reacted at 11% concentration in Isononyl Isononanoate (or another suitable cosmetically acceptable solvent). Once the elastomer gel is formed, the product is passed through a colloid mill and the concentration is then reduced to 6% by adding more solvent with good mixing until a uniform soft gel is formed.

Example 1 shows a preferred embodiment in which a water-in-oil (w/o) emulsion is formed using the hydrophilically modified silicone elastomer (A) that contains 10% w/w % of the elastomer polymer solids of a 10 mole ethoxylate of allyl alcohol. This product is known commercially as “NuLastic SURFA 10 D-99 6”. Example 2 is another preferred embodiment in which a w/o emulsion is formed using hydrophilic silicone elastomer (B) where the concentration of the ethoxylated allyl alcohol was decreased to 5% w/w of the elastomer polymer solids to make the elastomer less hydrophilic than the hydrophilic silicone elastomer (A). This product is known commercially as “NuLastic SURFA 5 D-99 6”. Glycerin is also added to this example to provide additional skin moisturization and to provide antimicrobial preservation for the formula. Also, Isododecane is used as the solvent instead of Isononyl Isononanoate to demonstrate the use of an alternate solvent. Example 3 again shows the use of the more hydrophilic silicone elastomer (A) that contains 10% w/w % of the polymer solids of the 10 mole ethoxylate of allyl alcohol. This example also shows the addition of a humectant to the water phase of the formulation to provide additional skin moisturization and antimicrobial preservation for the formula and the use of isododecane as the solvent. Such w/o emulsions as presented in the examples find use in cosmetics as moisturizing and emollient creams and lotions in skin care products and vehicles for water soluble materials such as, but not limited to: antiperspirants, skin lighteners, pH adjusters, reducing agents and oxidizing agents.

The following examples illustrate the use of a polyurethane modified hydrophilic silicone elastomer as an emulsifier.

| | Example 4 | Example 5 | Example 6 |
|---|---|---|---|
| Ingredients | % w/w | % w/w | % w/w |
| Isododecane | 23.5 | 18.0 | 63.0 |
| Hydrophilically modified silicone | 1.5 | xxx | 4.0 |
| elastomer (C) | | | |
| Hydrophilically modified silicone | xxx | 1.2 | xxx |
| elastomer (D) | | | |
| Water | 74.0 | 80.0 | 20.0 |
| Glycerin | xxx | xxx | 10.0 |
| Citric Acid (50% aq. solution) | 1.0 | 0.8 | 3.0 |
| | 100.0 | 100.0 | 100.0 |

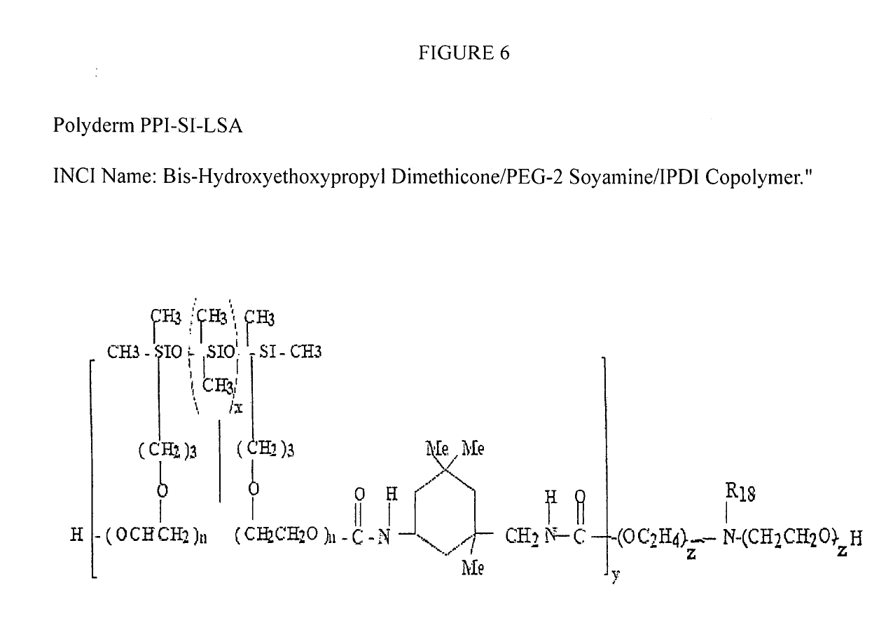

Example 4 shows a preferred embodiment in which a water-in-oil (w/o) emulsion is formed using the hydrophilically modified silicone elastomer (C) that contains about 33% w/w % of the elastomer polymer as a silicone/soyamine polyurethane made in the manner that are taught by U.S. Pat. No. 6,936,686 and otherwise described herein. The polyurethane is the product of the reaction between Isophorone Diisocyanate (IPDI) and a mixture of di-PEG-1 Polydimethylsiloxane diol and PEG-2 Soyamine. This product is known commercially as “Polyderm PPI-SI-LSA” (see FIG. 6). In this reaction, the unsaturated soy fatty amine reacts with the hydrogen terminated polydimethylsiloxane that is used as a crosslinking reactant to form the silicone elastomer. This incorporates the silicone/soyamine polyurethane into the silicone elastomer making the elastomer hydrophilic, particularly when the amine is protonated by adding an acid. Example 5 is another preferred embodiment in which a w/o is formed using the hydrophilic silicone elastomer (D). This elastomer also contains 33% w/w % of a polyurethane containing a silicone/soyamine polyurethane. Again, this polyurethane is the result of a reaction between Isophorone Diisocyanate and a combination of di-PEG-1 Polydimethylsiloxane diol and PEG-2 Soyamine. This product is known commercially as “Polyderm PPI-SI-LSAS” (modification of polyurethane in FIG. 6 to increase hydrophilicity). It differs from the silicone/soyamine polyurethane used in Hydrophilically modified silicone elastomer (C) by containing three times the amount of PEG-2 Soyamine thus, making the resulting silicone/soyamine polyurethane more hydrophilic. The unsaturated soy fatty amine reacts with the hydrogen terminated polydimethylsiloxane that is used as a crosslinking reactant to form the silicone elastomer. This incorporates the silicone/soyamine polyurethane into the silicone elastomer making the elastomer hydrophilic, especially when the amine is protonated by adding an acid. As seen in Example 5, the hydrophilically modified silicone elastomer (D) is capable of emulsifying more water than the hydrophilically modified silicone elastomer (C) that is used in Example 4. Example 6 shows the use of the less hydrophilically modified silicone elastomer (C) with a lower amount of water, but in combination with a humectant to form a w/o emulsion. These types of emulsions find use as a “base” and/or vehicle for active ingredients in skin care products such as, but not limited to: antiperspirants, skin lighteners, pH adjusters, reducing agents and oxidizing agents and as moisturizing, film forming and protective hand and body creams and lotions.

Further refinements include the use of a ricinoleic acid monoester of triethanolamine polymerized with bis-PEG-1 dimethicone and IPDI to make a polyurethane that is then copolymerized in the silicone elastomer in place of the urethane polymer described in elastomers C and D. Alternatively, oleic, palmitoleic, gadoleic, erucic, linoleic and linolenic acids may be substituted in whole or in part for the ricinoleic acid to make the initial triethanolamine monoester which is then further reacted with bis-PEG-1 dimethicone and IPIDI to make a urethane copolymer that is then copolymerized in the silicone elastomer in place of the urethane polymer described in elastomers C and D. Additionally, a copolyurethane that is composed of bis-PEG-1 dimethicone, castor oil, an unsaturated monoester of triethanolamine and or PEG-2 soyamine can be coreacted with the silicone elastomer. These copolyurethanes are easily prepared by one skilled in the art using well known methods for making the starting monoester, then adding a desired quantity of bis-PEG-1 dimethicone in such an amount that the amount of triethanolamine monoester constitutes from 1 to 50 mole percent of the bis-PEG-1 dimethicone and adding IPDI in an amount from 90 to 100 molar equivalent percent of the hydroxyl content.

The following examples illustrate the use of an alkylene-ethoxylate modified hydrophilic silicone elastomer as a delivery system and emulsifier for physiologically active materials.

| | Example 7 | Example 8 | Example 9 |
|---|---|---|---|
| Ingredients | % w/w | % w/w | % w/w |
| Isododecane | 61.1 | 61.1 | xxx |
| Dermol 99 (Isononyl Isononanoate) | xxx | xxx | 61.1 |
| Hydrophilically modified silicone | 3.9 | xxx | 3.9 |
| elastomer (A) | | | |
| Hydrophilically modified silicone | xxx | 3.9 | xxx |
| elastomer (B) | | | |
| Water | 10.0 | 10.0 | 10.0 |
| Propylene Glycol | 5.0 | 5.0 | 5.0 |
| Ethoxydiglycol | 10.0 | 10.0 | 10.0 |
| Dihydroxy Acetone (DHA) | 10.0 | 10.0 | 10.0 |
| | 100.0 | 100.0 | 100.0 |

These three Examples produce stable w/o emulsions that release DHA to effectively produce a tan color on skin.
The following example illustrates the use of an alkylene-ethoxylate modified hydrophilic silicone elastomer as a delivery system and as a vehicle to contain an ingredient until it can be activated by water. In this instance the hydrophilically modified silicone elastomer acts as a medium to allow and control the addition of water to the reactant.

| | Example 10 |
|---|---|
| Ingredients | % w/w |
| Isododecane | 70.5 |
| Hydrophilically modified silicone elastomer (A) | 4.5 |
| Zeolite 3A Powder | 25.0 |
| | 100.0 |

The addition of water to this system produces noticeable, measurable release of heat, due to the hydration of the Zeolite. This shows that the hydrophilically modified silicone elastomer is capable of allowing the transport of water to the Zeolite and that the Zeolite is maintained in a state that it is capable of reaction with water.
The following examples illustrate the use of an alkylene-ethoxylate modified hydrophilic silicone elastomer in various cosmetic and personal care products with applications as bases and vehicles for active ingredients and pigments.

| | Example 11 | Example 12 | Example 13 |
|---|---|---|---|
| Ingredients | % w/w | % w/w | % w/w |
| Part 1 | | | |
| Dermol 99 (Isononyl Isononanoate) | 18.8 | 32.9 | xxx |
| Isododecane | xxx | xxx | 23.5 |
| Hydrophilically modified silicone elastomer (A) | 1.2 | 2.1 | 1.5 |
| Glycerin | 2.0 | 10.0 | xxx |
| Water | 5.0 | 10.0 | xxx |
| Blue No. 1 (1% aq. Solution) | 0.1 | xxx | xxx |
| Pigment Blend (JC3-225) | xxx | xxx | 15.0 |
| Cab-O-Sil M-5 (Silica) | xxx | xxx | 1.0 |
| Part 2 | | | |
| Pemulen TR-1 (Acrylates/C10-30 Alkyl Acrylate | 0.2 | xxx | xxx |
| Crosspolymer) | | | |
| Water | 72.3 | xxx | 52.5 |
| Butylene Glycol | xxx | xxx | 3.0 |
| Veegum F (Magnesium Aluminum Silicate) | xxx | xxx | 1.2 |
| Keltrol T (Xanthan Gum) | xxx | xxx | 0.6 |
| Ceteth 20 | xxx | xxx | 1.0 |
| Polyderm PPI-SI-WS (Bis-PEG-15 Dimethicone/ | xxx | xxx | 0.5 |
| IPDI Copolymer) | | | |
| Triethanolamine | 0.2 | xxx | xxx |
| Methylisothiazolinone (1.5% aq. solution) | 0.2 | xxx | 0.2 |
| Dermol DGDIS (Polyglyceryl-2 Diisostearate) | xxx | 10.0 | xxx |
| Castor Oil | xxx | 5.0 | xxx |
| Pigment Blend (JC3-273) | xxx | 12.0 | xxx |
| Performalene (Polyethylene) | xxx | 6.0 | xxx |
| Performacol 550 (C30-50 Alkyl Alcohols) | xxx | 8.0 | xxx |
| SP 89 (Microcrystalline Wax) | xxx | 2.0 | xxx |
| SP 1020 (Ozokerite Wax) | xxx | 2.0 | xxx |
| | 100.0 | 100.0 | 100.0 |

Example 11 is a water-in-oil-in-water (w/o/w) emulsion made by first mixing part 1 and, once that is uniform, adding it to the part 2 mixture with good mixing. This Example serves as a moisturizing hand and body lotion as well as a base for other products for example; make-up products or products with water soluble active ingredients that can be incorporated in the central internal water phase. Additional cosmetic esters, sunscreens, oils, dimethicones, cyclomethicones and hydrocarbons can be added to part 1 to modify its feel, volatility, function and other cosmetic properties. Example 12 is an example of a lipstick incorporating water and a humectant in an internal phase. The moisturizing ingredients (water and glycerin) are released upon application to the lips. Colorants, flavors and active ingredients can also be added to the internal water phase. The product is made by combining part 1 with good mixing until uniform and then heating, with continued mixing, to approximately 90° C. Part 2 is combined and heated with mixing to approximately 110° C. Once the waxes are melted, part 2 is cooled to about 100° and part 1 is added with good mixing. Mixing is continued and the batch is cooled to about 85° C. and poured into molds. In addition to the stick format, Example 12 can be easily made into a lip gloss by deletion of the waxes (Performalene, Performacol, SP 89 and SP 1020) with a concomitant increase in Dermol DGDIS, or the addition Polyderm PPI DGDIS (a polyurethane made from the reaction of Polyglyceryl-2 Diisostearate and IPDI), or Polyderm PPI CO (a polyurethane made from the reaction of Castor Oil and IPDI), or the like. Of course, once the waxes are deleted, heating is no longer necessary. Example 13 is a make-up formula that remarkably easily incorporates caprylsilane treated pigments treated into a water based emulsion. This Example combines the elegant dry feel and smooth application of an elastomer with the uniform color deposition, stability and economy of a typical oil-in-water (o/w) emulsion type make-up. In this Example the silicone elastomer acts as a the low HLB emulsifier and oil phase component of the emulsion helping to both wet the caprylsilane treated pigments and to form a stable interface with the water phase. Similar formulations, with appropriate pigmentation can be used as rouges, mascaras and eyeliners.

The incorporation of sunscreen actives, especially the particulate types, can be problematic and can produce products that are unaesthetic. The following examples illustrate the use of an alkylene-ethoxylate modified hydrophilic silicone elastomer to produce aesthetically pleasing sunscreen products.

| | Example 14 | Example 15 | Example 16 |
|---|---|---|---|
| Ingredients | % w/w | % w/w | % w/w |
| Part 1 | | | |
| Dermol 99 (Isononyl Isononanoate) | 18.8 | 14.1 | 18.8 |
| Isododecane | 18.8 | 18.8 | 18.8 |
| Hydrophilically modified silicone elastomer (A) | 2.4 | 2.1 | 2.4 |
| T Cote 031 | 10.0 | xxx | 10.0 |
| Z Cote HP-1 | xxx | 10.0 | xxx |
| Octyl Methoxycinnamate | xxx | 7.5 | xxx |
| Part 2 | | | |
| Pemulen TR-1 (Acrylates/C10-30 Alkyl Acrylate Crosspolymer) | 0.2 | xxx | 0.1 |
| Water | 49.4 | 44.8 | 34.8 |
| Dermothix 100 (Steareth 100 IPDI) | xxx | 2.0 | xxx |
| Triethanolamine | 0.2 | xxx | 0.1 |
| Methylisothiazolinone (1.5% aq. solution) | 0.2 | 0.2 | 0.2 |
| Polyderm PEPA | xxx | xxx | 15.0 |
| Castor Oil | xxx | xxx | xxx |
| Pigment Blend (JC3-273) | xxx | xxx | xxx |
| | 100.0 | 100.0 | 100.0 |

Example 14 shows a stiff or high viscosity cream made with the hydrophilic silicone elastomer and a physical sunscreen. The use of the hydrophilic silicone elastomer allows for the incorporation of the silicone treated TiO₂ into part 1 and then the incorporation of it into the aqueous phase (part 2). In addition, the elastomer gives the cream a smooth, elegant not greasy or draggy application. Example 15 uses two sunscreen agents; one particulate and the other an organic UV absorbing ester. Part 1 of this formula is a smooth, white, opaque, viscous liquid which is easily added to part 2. Example 16 shows the addition of a film forming ingredient (Polyderm PEPA) to a hydrophilic silicone elastomer based sunscreen cream to improve the abrasion resistance and reduce the water wash off.
The following examples show the use of hydrophilic silicone elastomers in surfactant based products such as shampoos and hair conditioners.

| | Example 17 | Example 18 |
|---|---|---|
| Ingredients | % w/w | % w/w |
| Part 1 | | |
| Water | 47.8 | 69.3 |
| Veegum F (Magnesium Aluminum Silicate) | 1.0 | xxx |
| Keltrol T (Xanthan Gum) | 0.5 | xxx |
| Ceteth 20 | xxx | 1.2 |
| Cetearyl Alcohol | xxx | 3.8 |
| Nequat DBS (Stearyl Alcohol & Stear- | xxx | 5.0 |
| amidopropyl Ethyldimonium Ethosulfate & | | |
| Dimethyl Lauramine & Dilinoleic Acid | | |
| Part 2 | | |
| Ammonium Laureth (3) Sulfate | 15.0 | xxx |
| Ammonium Lauryl Sulfate | 15.0 | xxx |
| Cocamidopropyl Hydroxysultaine | 10.0 | xxx |
| Isododecane | 9.4 | xxx |
| Dermol 99 (Isononyl Isononanoate) | xxx | 18.8 |
| Hydrophilically modified silicone | 0.6 | 1.2 |
| elastomer (A) | | |
| Fragrance | 0.5 | 0.5 |
| Methylisothiazolinone (1.5% aq. | 0.2 | 0.2 |
| solution) | | |
| | 100.0 | 100.0 |

Example 17 is a hair shampoo using the of hydrophilic silicone elastomer as a hair conditioning agent that precipitates from the shampoo upon dilution. Example 18 shows the use of a hydrophilic silicone elastomer in a hair conditioner where it provides greatly enhanced conditioning and shine to the hair.

What is claimed:

1. A hydrophilic silicone elastomer comprising the reaction product of a polyorganosiloxane polymer which is reacted with an allyl alcohol ethoxylate and/or a polyurethane polymer.

2. The hydrophilic silicone elastomer according to claim 1 wherein said polyorganosiloxane is first reacted with a second polyorganosiloxane polymer before being reacted with said allyl alcohol ethoxylate and/or a polyurethane polymer.

3. The hydrophilic silicone elastomer according to claim 1 wherein said allyl alcohol ethoxylate is according to the chemical structure: 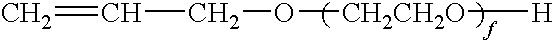
Where f is a positive integer from 1 to 20.

4. The hydrophilic silicone elastomer according to claim 1 wherein said polyorganosiloxane polymer is a polymeric composition comprised of 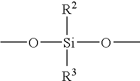
units, where R² and R³ are independently H, OH, C₁-C₁₀ alkyl, C₂-C₁₀ alkene or aryl groups and varies in average molecular weight Mₙ from about 1,000 to about 1,500,000 or more, preferably about 1,000 to about 100,000.

5. The hydrophilic silicone elastomer according to claim 4 wherein said polyorganosiloxane polymer contains a number of groups which are capable of crosslinking with an allyl alcohol ethoxylate according to the chemical structure: 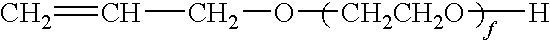
Where f is a positive integer from 1 to 20; and/or
with a reactive polyurethane polymer containing a crosslinkable functional group.

6. The hydrophilic silicone elastomer according to claim 5 wherein said reactive polyurethane polymer contains a hydrogen, OH, NH₂ or olefinic functional group.

7. The hydrophilic silicone elastomer according to claim 5 wherein said polyorganosilixane polymer contains a number of groups which are capable of crosslinking with said alkyl alcohol ethoxylate and said polyurethane polymer.

8. The hydrophilic silicone elastomer according to claim 4 wherein said polyorganosiloxane polymer is a compound according to the chemical structure: 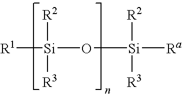
Where R¹ and R^{a} are independently H, a C₁-C₆ alkyl group which is optionally substituted with OH or a C₁-C₃ alkyl group which itself is optionally substituted with a hydroxyl group or an optionally substituted C₂-C₆ alkenyl group;
Each R² and R³ is independently H, OH, or a C₁-C₃ alkyl group; and
n is from 5 to 50,000.

9. The hydrophilic silicone elastomer according to claim 8 wherein said alkenyl group is an acrylate or (meth)acrylate group.

10. The hydrophilic silicone elastomer according to claim 1 wherein said polyorganosiloxane polymer is a compound according to the chemical structure: 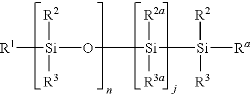
Where R¹ and R^{a} are each independently H, a C₁-C₆ alkyl group optionally substituted with OH or a C₁-C₃ alkyl group which itself is optionally substituted with a hydroxyl group, or an optionally substituted C₂-C₆ alkenyl group;
Each R² and R³ is independently H, OH, or a C₁-C₃ alkyl group;
Each R^{2a} and R^{3a} is independently H, OH, or a C₁-C₃ alkyl group;
n is from 5 to 50,000; and
j is from 0 to 50.

11. The hydrophilic silicone elastomer according to claim 1 wherein said polyorganosiloxane polymer is crosslinked with a polysiloxane crosslinking agent before reaction with said allyl alcohol ethoxylate and/or polyurethane.

12. The hydrophilic silicone elastomer according to claim 1 wherein said polyurethane has the general structure: 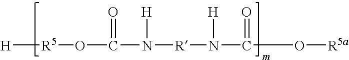
Where R⁵ is a C₁-C₅₀ hydrocarbon or a siloxane group each of which groups may be optionally substituted with at least one hydroxyl and/or polyethylene glycol group and wherein each of said hydrocarbon or siloxane groups optionally contains at least one polymerizable olefinic group;
R^{5a} is a C₁-C₅₀ hydrocarbon or a siloxane group each of which groups may be optionally substituted with hydroxyl and/or polyethylene glycol groups and wherein each of said hydrocarbon or siloxane groups optionally contains at least one polymerizable olefinic group;
R′ is an optionally substituted C₂ through C₃₆ linear, cyclic or branch-chained saturated or unsaturated hydrocarbon group; and m is from 1 to about 250.

13. The hydrophilic silicone elastomer according to claim 12 wherein R⁵ is a C₂-C₅₀ hydrocarbon group containing at least one olefinic group which is optionally substituted with at least one hydroxyl group and/or a polyethylene glycol group wherein said polyethylene glycol group ranges in size from 1 to about 100 polyethylene oxide groups, or a polyethylene oxide group comprising between 1 and 500 ethylene oxide groups which is endcapped with a polymerizable alkenyl, acrylate or (meth)acrylate group and R^{5a} is a C₂-C₅₀ hydrocarbon group containing at least one olefinic group which is optionally substituted with hydroxyl groups and/or polyethylene glycol groups, or a polyethylene oxide group comprising between 1 and 500 ethylene oxide groups which is endcapped with a polymerizable alkenyl, acrylate or (meth)acrylate group and R′ is an isophorone group.

14. The hydrophilic silicone elastomer according to claim 12 wherein at least one of R⁵ and R^{5a} is a siloxane group and wherein said R⁵ siloxane group has the structure: 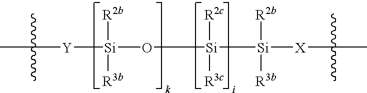
and
said R^{5a} siloxane group has the structure: 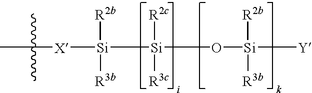
Wherein Y is absent, O or a —W—(O—Z)ᵣ-Q-(CH₂)_{q}-T-group;
X is absent or a -TCH₂)_{q}-Q-(Z—O)ᵣ—W′-group;
X′ is absent or a —W′—(O—Z)ᵣ-Q-(CH₂)_{q}-T-group;
Y′ is absent or a -TCH₂)_{q}-Q-(Z—O)ᵣ—W² group;
Y′ is absent or a group;
W is absent when r is an integer of 1 or more and W is absent or O when r is 0;
Q is absent or O;
q is an integer from 0 to 10;
r is an integer from 0 to 100, with the proviso that q or r is at least 1;
T is absent or O;
W′ is absent when r is 0 and is a Z group when r is 1 or more;
W² is H;
Z is independently an ethylene group, a propylene group or a mixture of ethylene and propylene groups;
R^{2b} and R^{3b} are each independently H or a C₁-C₃ alkyl group;
R^{2c} and R^{3c} are independently selected from H, an optionally substituted C₁-C₆ alkyl group or an optionally substituted C₂-C₆ alkenyl group;
i is an integer from 0 to 50; and
k is an integer from 0 to 100.

15. The hydrophilic silicone elastomer according to claim 14 wherein R⁵ is a O—R⁶ group and R^{5a} is a R⁶—OH group wherein R⁶ is a C₁-C₅₀ hydrocarbon which is optionally substituted with at least one hydroxyl group and/or polyethylene glycol groups ranging in size from about 2 to about 100 polyethylene oxide units,

16. The hydrophilic silicone elastomer according to claim 1 in the form of a gel.

17. The hydrophilic silicone elastomer according to claim 1 wherein hydrophilic character is instilled in said elastomer by incorporation of effective amounts of allyl alcohol ethoxylate residues.

18. The hydrophilic silicone elastomer according to claim 1 wherein hydrophilic character is instilled in said elastomer by incorporation of effective amounts of allyl alcohol ethoxylate residues and/or a polyurethane residue.

19. The hydrophilic silicone elastomer according to claim 1 in combination with an oil.

20. The hydrophilic silicone elastomer according to claim 1 in combination with a preservative, an antioxidant or mixtures thereof.

21. An emulsion comprising an effective amount of a hydrophilic silicone elastomer according to claim 1 in combination with an oil, water and optionally, a secondary emulsifier (a traditional emulsifier).

22. The emulsion according to claim 21 further comprising at least one additional component selected from the group consisting of an aqueous solvent, a non-aqueous solvent, emollients, humectants, oils, conditioning agents, emulsifiers, surfactants, thickeners, stiffening agents, medicaments, fragrances, preservatives, deodorant components, anti-perspirant compounds, skin protecting agents, pigments, vitamins, nutraceuticals, sunscreens and mixtures thereof.

23. A personal care composition comprising a hydrophilic silicone elastomer or emulsion composition according to claim 1.

24. The personal care composition according to claim 23 wherein said additional component selected from the group consisting of an aqueous solvent, a non-aqueous solvent, emollients, humectants, oils, conditioning agents, emulsifiers, surfactants, thickeners, stiffening agents, medicaments, fragrances, preservatives, deodorant components, anti-perspirant compounds, skin protecting agents, pigments, vitamins, antioxidants, sunscreens and mixtures thereof.

25. A method of preparing a hydrophilic silicone elastomer according to claim 1 by reacting a polyorganosiloxane with about 0.5% to about 25% by weight of the reaction mixture of an allyl alcohol ethoxylate monomer and/or a polyurethane in a single step reaction.

26. The method according to claim 25 wherein said allyl alcohol ethoxylatre and/or said polyurethane comprises about 1% to about 15% by weight of the reaction mixture which includes silicone elastomer, allyl alcohol ethoxylate and/or polyurethane and any additional reactants included.

27. The method according to claim 25 wherein said allyl alcohol ethoxylatre and/or said polyurethane comprises about 1% to about 10% by weight of the reaction mixture which includes silicone elastomer, allyl alcohol ethoxylate and/or polyurethane and any additional reactants included.

28. A method of preparing an emulsion comprising mixing a hydrophilic silicone elastomer according to claim 1 with an oil, water and optionally, a secondary emulsifier and optionally, at least one additional component selected from the group consisting of aqueous solvent (e.g. alcohol or other water compatible solvent), a non-aqueous solvent, emollients, humectants, oils (polar and non-polar), conditioning agents, emulsifiers, including secondary emulsifiers, surfactants, thickeners, stiffening agents, medicaments, fragrances, preservatives, deodorant components, anti-perspirant compounds, skin protecting agents, pigments, sunscreens and mixtures thereof.

29. A method of instilling hydrophilic character to a silicone elastomer comprising reacting a polyorganosiloxane compound with a polyurethane and/or allyl alcohol ethoxylate to produce a hydrophilic silicone elastomer.
